(19) United States
(12) Patent Application Publication
(10) Pub. No.: US 2019/0371454 A1
(43) Pub. Date: Dec. 5, 2019

(54) MEDICAL IMAGE INFORMATION STORAGE SYSTEM

(71) Applicant: Coreline Soft Co., Ltd, Seoul (KR)

(72) Inventors: Donghoon Yu, Seoul (KR); Jaeyoun Yi, Seoul (KR)

(73) Assignee: CORELINE SOFT CO., LTD.

(21) Appl. No.: 16/431,018

(22) Filed: Jun. 4, 2019

(30) Foreign Application Priority Data
Jun. 4, 2018 (KR) 10-2018-0064259

(51) Int. Cl. G16H 30/20; G16H 30/40

(52) U.S. Cl. CPC G16H 30/20; G16H 10/60; G16H 30/40

(57) ABSTRACT

Disclosed is a system for storing and transferring medical images, the system including a medical image acquisition unit adapted to acquire at least one medical image, an operation unit adapted to analyze the at least one medical image and to generate a connecting image, with the connecting image being generated to connect the at least one medical image and corresponding analysis information and being inserted into the at least one medical image, a server adapted to transfer the at least one medical image having the connecting image inserted therein, and a display adapted to display the at least one medical image having the connecting image inserted therein.

### CROSS REFERENCE TO RELATED APPLICATION

This application claims the benefit of and priority to Korean Patent Application No. 10-2018-0064259, filed on Jun. 4, 2018. The entire disclosure of the above application is incorporated herein by reference.

### FIELD

The present disclosure is generally related to a system for storing and transferring medical images, and in particular, to a system for storing and transferring medical images that promotes convenient connection between images and information.

### BACKGROUND

This section provides background information related to the present disclosure which is not necessarily prior art.

FIG. 1 shows an example of a DICOM (Digital Imaging and Communication in Medicine) image relay system for use in a system for storing and transferring medical images presented in Korean Patent Application Laid-Open No. 10-2007-0022975.

The DICOM is involved in storing and transferring medical image data within the system.

With technical advances in electronic engineering, a number of medical devices have been introduced into the market. For instance, computerized tomography (CT) and magnetic resonance imaging (MRI) devices are readily available. With these devices, it is now possible to make highly accurate diagnoses of many diseases or disorders that were nearly impossible to be properly diagnosed in the past.

In addition, diagnostic imaging devices have been remarkably improved in recent years, showing a sharp increase in use of radiation in diagnosis. Today, approximately 50-70% of patients including both inpatients and outpatients take radiology examinations, and get proper treatment regimens recommended for them based on the examination results. As hospitals or clinics are getting bigger, more systematic and more efficient, the availability of a high-efficiency medical diagnostic or examination imaging service and radiology specialty doctors who would access and diagnose a patient's condition by reading imaging scans and quickly transfer the results to the patient's physician has also become very critical.

Examples of diagnostic imaging technologies may include CT scans, MRIs, X-rays, Ultrasound (US) scans, endoscopy, angiography, colposcopy, cervicography, PET scans, SPECT, ECG, EEG, MEG and so forth, which are broadly used in the departments of internal medicine, general surgery, or obstetrics. However, when a patient desires to have any diagnostic imaging exam, there are many hospitals without proper imaging equipment, and there are many hospitals with proper imaging equipment but without radiology specialty doctors available to read medical images. Even if there are hospitals where both proper imaging equipment and radiology specialty doctors are available, it is not easy to squeeze into exam schedules already filled up. Either a patient's clinic having no medical imaging equipment and no radiologist, or a patent's clinic having only medical imaging equipment without a radiologist's employment, the patient is given a referral to make an appoint at other hospitals and imaging facilities to get a desired imaging exam or test. This procedure is inefficient in terms of time and cost on a patent as well as the patient's physician who have to wait for medical imaging data and its reading result in a written report.

Especially when a radiology specialty doctor is not available, a hospital or clinic might send a patient's medical imaging data that the patient has obtained from a diagnostic imaging center to an intermediary agent who would find a radiology outsourcing company that provides an expedient radiology interpretation and reading service of medical images. The reading results are then sent to the patient's referring physician via the agent. Unfortunately, this entire process was not done via electronic communication, increasing more burden on patients and their health carers in timewise and moneywise.

Many hospitals now have a picture archiving communication system (PACS) for storing and transferring medical images. In PACS, a computer communication network is established throughout the entire hospital, and every X-ray image is converted into digital data to build a database. The data is then stored in a mass storage medium connected to a server. Thus, whenever needed, a patient's physician can search for X-ray images of his patient and see them directly on his own computer monitor at his office. Nevertheless, this PACS is not yet efficiently used.

For instance, in an emergency room, computed radiography (CR) or CT scanning is carried out on an emergency patient, and the resulting images can be seen immediately with a PACS display workstation in the emergency room.

However, the current PACS system stores those scanned images separately from other information on a patient, e.g., the patient's personal information, medical history and a pre-existing disease or condition, making it inconvenient for physicians or health care providers. Therefore, there exists a need to develop ways for easy and quick access to a patient's personal information, medical history, a pre-existing disease or condition and diagnostic imaging data all together.

### SUMMARY

This section provides a general summary of the disclosure and is not a comprehensive disclosure of its full scope or all of its features.

According to one aspect of the present disclosure, there is provided a system for storing and transferring medical images, the system including: a medical image acquisition unit adapted to acquire at least one medical image, an operation unit adapted to analyze the at least one medical image and to generate a connecting image, with the connecting image being generated to connect the at least one medical image and corresponding analysis information and being inserted into the at least one medical image, a server adapted to transfer the at least one medical image having the connecting image inserted therein, and a display adapted to display the at least one medical image having the connecting image inserted therein.

These and other advantages will be apparent to those skilled in the art in view of the present drawings and specification.

### BRIEF DESCRIPTION OF THE DRAWINGS

For a more complete understanding of the present disclosure and the advantages thereof, reference is now made to the following description taken in conjunction with the accompanying drawings in which like reference numerals indicate like features and wherein:

FIG. 1 illustrates an exemplary DICOM (Digital Imaging and Communication in Medicine) image relay system for use in a system for storing and transferring medical images presented in Korean Patent Application Laid-Open No. 10-2007-0022975;

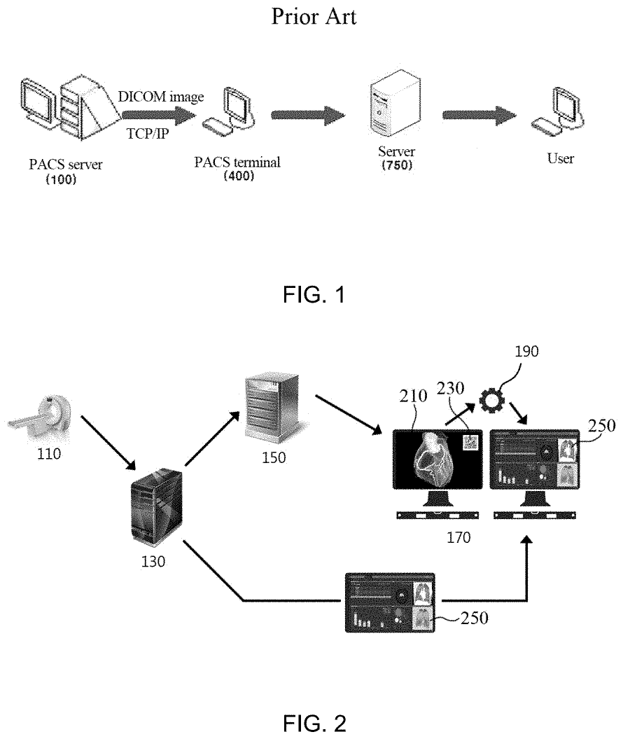

FIG. 2 illustrates an exemplary embodiment of a system for storing and transferring medical images, according to the present disclosure;

FIG. 3 illustrates an exemplary method for adding a connecting image to medical images, according to the present disclosure;

FIG. 4A and FIG. 4B illustrate a medical image and an analyzed image, respectively, according to the present disclosure; and

FIG. 5A and FIG. 5B illustrate examples of inserting a connecting image, according to the present disclosure.

### DETAILED DESCRIPTION

The present disclosure will now be described in detail with reference to the accompanying drawing(s).

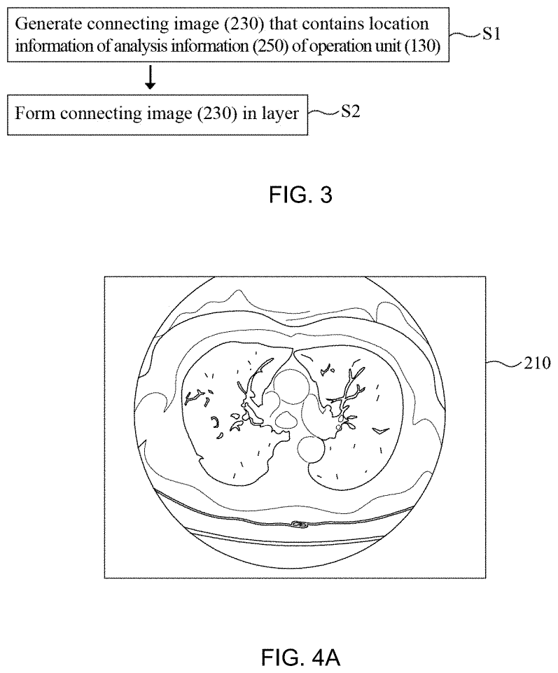

FIG. 2 illustrates an exemplary embodiment of a system for storing and transferring medical images, according to the present disclosure.

The system for storing and transferring medical images includes a medical image acquisition unit 110, an operation unit 130, a server 150, and a display 170. The medical image acquisition unit 110 acquires medical images 210 either by taking images of a patient or by retrieving previously taken medical images 210. For instance, the medical image acquisition unit 110 utilizes a CT, MRI, X-ray, Ultrasound, PET, SPECT, ECG, EEG, MEG or endoscopy machine or equipment, in order to obtain medical images 210. The medical images 210 may include all medical images including those used in dental and ophthalmic practices. Besides the machines mentioned above, any medical imaging device can be used to obtain such medical images 210. The medical image acquisition unit 110 can take at least one medical image 210. The at least one medical image 210 of a patient can be combined into data series for transmission.

The operation unit 130 analyzes the medical images 210 obtained by the medical image acquisition unit 110. The operation unit 130 stores the medical images 210 from the medical image acquisition unit 110 as well as their analysis information 250 in its database.

The analysis information 250 and the medical images 210 are separately stored such that one must search for a medical image 210 of interest first and then its corresponding analysis information 250. To make things simpler, the operation unit 130 generates a connecting image 230 that connects the medical image 210 to its analysis information 250. In particular, a connecting image 230 in PACS connects a medical image 210, analysis information 250 thereof, and patient's information (including personal information, medical history, a pre-existing disease or condition, etc.), without modifying the PACS.

As discussed above, the connecting image 230 connects the medical image 210 and its analysis information 250. The connecting image 230 can include location information (e.g., URL information) on a position where the analysis information 250 is stored. As such, the location information can be used to fetch the desired analysis information 250. Moreover, the connecting image 230 can comprise ID information including, for example, patient ID, study number, series number and analysis items (e.g., LAA analysis).

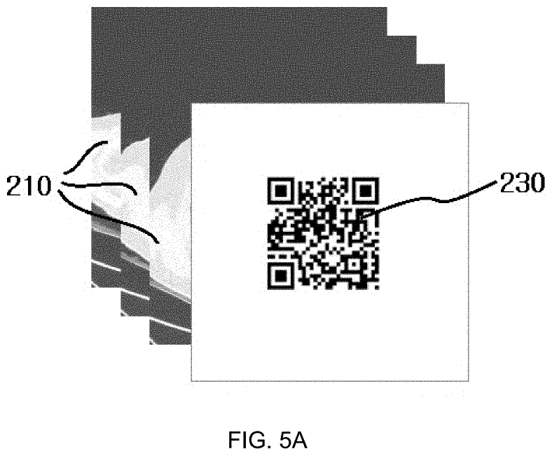

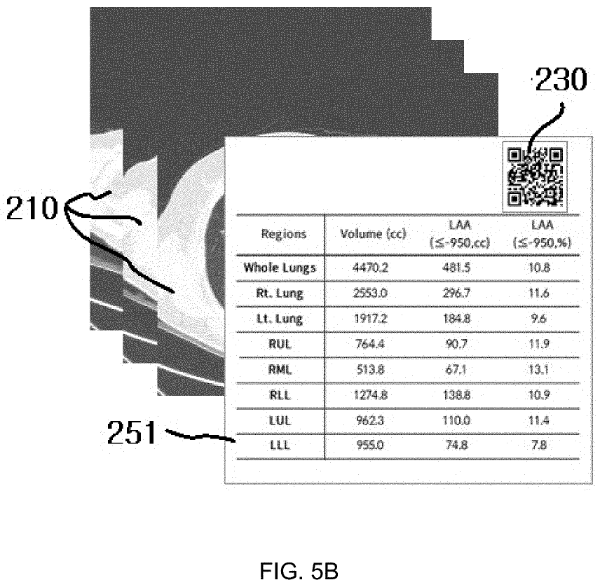

The connecting image 230 is created in an image file similar to the medical image 210, and may be inserted as a part of the medical image 210. The connecting image 230 can output the analysis information 250 obtained from the medical image 210, and generate an analysis image 251 (see FIG. 4(B)) based on the analysis information 250. The connecting image 230 can be added to the analysis image 251 and transferred together. This analysis image 251 may include a table or graph. More details on this will be provided below in reference to FIG. 4 and FIG. 5.

At least one medical image 210 can be provided, and the connecting image 230 can be inserted into one of the at least one medical image 210. Similarly, the analysis image 251 can be inserted into one of the at least one medical image 210.

The connecting image 230 is generated by converting location information of the operation unit 130 where the analysis information 250 is stored into an image. For instance, the connecting image 230 is formed of a QR code, and an analysis software executed in the operation unit 130 is configured to be connected to a thin client (this is a client hardware aiming for reducing TCO (Total Cost of Ownership) in a client/server system), allowing for connection simply with a URL address.

The server 150 transfers the medical image 210, together with the connecting image 230 inserted therein. This medical image 210 may further include a graph generated based on the analysis information 250. For instance, a system for storing and transferring medical image information actually available in a hospital comprises the medical image acquisition unit 110, the cover 150, and the display 170. This system for storing and transferring medical image information is involved in storing, transferring and displaying the medical image 210.

The display 170 displays the medical image 210 with the connecting image 230 inserted therein. The display 170 can be any of monitors or smart devices, which can present a screen.

The system for storing and transferring medical images may include a load unit 190, which recognizes the connecting image 230 inserted in the displayed medical image 210 and fetches the corresponding analysis information 250 from the operation unit 130. The load unit 190 can be a program or App, and brings necessary information by recognizing the connecting image 230. It can recognize the connecting image 230 with an external program or App, and fetch location information of the analysis information 250 through the connecting image 230. Here, the connecting image 230 may include ID information including, for example, patient ID, study number, series number and analysis items (e.g., LAA analysis). The load unit 190 can search the server 150 for ID information and output the corresponding ID information onto the display 170.

The DICOM standard in the medical image 210 is a generic term for the services and protocols for use in digital imaging and communications in medical devices. Needless to say, in the system for storing and transferring medical images according to the present disclosure, data telecommunication, data storage and data transfer are done to conform to the DICOM standard.

In particular, the load unit 190 and the operation unit 130 are newly added components to the conventional system for storing and transferring medical images. With both units being connected to the conventional system for storing and transferring medical images, the analysis information 250 can be fetched using the connecting image 230.

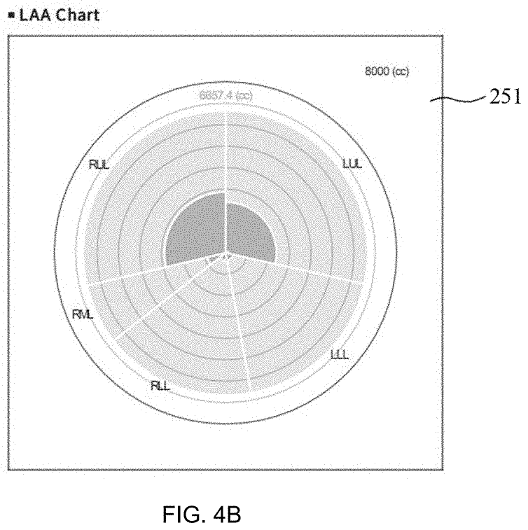

FIG. 3 is a flow chart illustrating an exemplary method of inserting a connecting image into a medical image, according to the present disclosure.

In order to insert a connecting image 230 into a medical image 210, first, the operation unit 130 generates a connecting image 230 containing at least one of location information or ID information of the analysis information 250 of the operation unit 130. Next, the operation unit 130 forms the connecting image 230 in a layer. This layer of the connecting image 230 thus generated can then be inserted into the medical image 210. Here, the connecting image 230 is inserted into the medical image 210 such that it is not overlapped with the medical image 210.

FIG. 4 illustrates exemplary medical and analysis images according to the present disclosure.

In particular, FIG. 4(A) illustrates an example of a thoracic radiograph medical image 210.

FIG. 4(B) illustrates an analysis image 251 of the thoracic radiograph. The analysis image 251 can be generated based on the analysis information 250.

FIG. 5 illustrates examples of inserting the connecting image according to the present disclosure.

In particular, FIG. 5(A) shows that the connecting image 230 is inserted in the very front of at least one medical image 210.

FIG. 5(B) shows that the analysis information 250 in form of a graph is provided at the very front of at least one medical image 210, and the connecting image 230 is inserted such that it is not overlapped with the analysis information 250.

For instance, the operation unit 130 analyzes a plurality of medical images 210 and generates a connecting image 230. Analysis information 250 is used for generating an analysis image 251. The analysis image 251 is placed at the very front of the plurality of medical images 210, and then the connecting image 230 is placed such that it is not overlapped with the analysis image 251.

Set out below are a series of clauses that disclose features of further aspects of the invention, which may be claims.

(1) A system for storing and transferring medical images, the system including: a medical image acquisition unit adapted to acquire at least one medical image; an operation unit adapted to analyze the at least one medical image and to generate a connecting image, with the connecting image being generated to connect the at least one medical image and corresponding analysis information and being inserted into the at least one medical image; a server adapted to transfer the at least one medical image having the connecting image inserted therein; and a display adapted to display the at least one medical image having the connecting image inserted therein.

(2) There is also provided, the system of clause (1) wherein: the at least one medical image comprises a plurality of images, and the connecting image is inserted into one of the plurality of images.

(3) There is also provided, the system of clause (1) wherein: the connecting image is inserted into a part of one of the at least one medical image.

(4) There is also provided, the system of clause (1) wherein: the operation unit comprises an analysis image generated based on the analysis information, and the analysis image is inserted into one of the at least one medical image, with the connecting image being inserted into a part of the analysis image.

(5) There is also provided, the system of clause (1) wherein: the connecting image comprises at least one of location information or ID information of the analysis information.

(6) There is also provided, the system of clause (5) wherein: the connecting image is a QR code.

(7) There is also provided, the system of clause (1) further including: a load unit adapted to recognize the connecting image displayed on the display unit and to fetch analysis information from the operation unit.

(8) There is also provided, the system of clause (7) wherein: the load unit fetches a web address where the analysis information of the operation unit is stored.

(9) There is also provided, the system of clause (8) wherein: the load unit displays the analysis information.

(10) There is also provided, the system of clause (3) wherein: the connecting image is inserted into the at least one medical image such that the connecting image is not overlapped with the medical image.

(11) There is also provided, the system of clause (1) wherein: the at least one medical image is prepared in an image file.

(12) There is also provided, the system of clause (1) wherein: the at least one medical image and the connecting image are prepared in image files.

(13) There is also provided, the system of clause (1) wherein: the operation unit stores patient's information, and the load unit fetches the patient's information from the operation unit.

The system for storing and transferring medical images according to an exemplary embodiment of the present disclosure utilizes a connecting image to make it possible to see medical images and their analysis information all at once, without modifying the existing system.

The system for storing and transferring medical images according to another exemplary embodiment of the present disclosure makes it possible to fetch medical images as well as their analysis information more efficiently through the operation unit and the load unit incorporated into the existing system.

What is claimed is:

1. A system for storing and transferring medical images, the system comprising:
a medical image acquisition unit adapted to acquire at least one medical image;
an operation unit adapted to analyze the at least one medical image and to generate a connecting image, with the connecting image being generated to connect the at least one medical image and corresponding analysis information and being inserted into the at least one medical image;
a server adapted to transfer the at least one medical image having the connecting image inserted therein; and
a display adapted to display the at least one medical image having the connecting image inserted therein.

2. The system according to claim 1, wherein the at least one medical image comprises a plurality of images, and the connecting image is inserted into one of the plurality of images.

3. The system according to claim 1, wherein the connecting image is inserted into a part of one of the at least one medical image.

4. The system according to claim 1, wherein the operation unit comprises an analysis image generated based on the analysis information, and the analysis image is inserted into one of the at least one medical image, with the connecting image being inserted into a part of the analysis image.

5. The system according to claim 1, wherein the connecting image comprises at least one of location information or ID information of the analysis information.

6. The system according to claim 5, wherein the connecting image is a QR code.

7. The system according to claim 1, further comprising:
a load unit adapted to recognize the connecting image displayed on the display unit and to fetch analysis information from the operation unit.

8. The system according claim 7, wherein the load unit fetches a web address where the analysis information of the operation unit is stored.

9. The system according to claim 8, wherein the load unit displays the analysis information.

10. The system according to claim 3, wherein the connecting image is inserted into the at least one medical image such that the connecting image is not overlapped with the medical image.

11. The system according to claim 1, wherein the at least one medical image is prepared in an image file.

12. The system according to claim 1, wherein the at least one medical image and the connecting image are prepared in image files.

13. The system according to claim 1, wherein the operation unit stores patient's information, and the load unit fetches the patient's information from the operation unit
